(19) United States
(12) Patent Application Publication
(10) Pub. No.: US 2017/0196789 A1
(43) Pub. Date: Jul. 13, 2017

(54) REDUCTION OF ORAL AND EPIDERMAL MALODOR IN HUMANS AND ANIMALS

(71) Applicants: Artin Barzgar, Woodbridge, VA (US); Manoucherhr Kiani, Zaferanieh, Tehran (IR); Sepideh Kiani, Zaferanieh (IR)

(72) Inventors: Artin Barzgar, Woodbridge, VA (US); Manoucherhr Kiani, Zaferanieh, Tehran (IR); Sepideh Kiani, Zaferanieh (IR)

(21) Appl. No.: 15/321,291

(22) PCT Filed: Jun. 26, 2015
(86) PCT No.: PCT/US15/37954

§ 371 Date: Dec. 22, 2016

Related U.S. Application Data
(60) Provisional application No. 62/017,314, filed on Jun. 26, 2014.

(51) Int. Cl. A61K 8/43; A61K 8/24; A61K 8/34; A61K 8/73; A61K 8/49; A61K 8/29; A61K 8/37; A61K 9/00; A61Q 11/00; A61K 8/06; A61K 9/06; A61K 31/155; A61K 31/315; A61Q 19/10; A61K 8/58; A61Q 15/00; A61K 8/25

(52) U.S. Cl. CPC A61K 8/43; A61K 8/25; A61K 8/24; A61K 8/345; A61K 8/731; A61K 8/49; A61K 8/34; A61K 8/29; A61K 8/37; A61K 9/0043; A61Q 11/00; A61K 8/06; A61K 9/06; A61K 9/0014; A61K 31/155; A61K 31/315; A61Q 19/10; A61K 8/58; A61Q 15/00; A61K 2800/87; A61K 2800/5922

(57) ABSTRACT

Some methods for preventing or reducing oral and epidermal malodor by the use of formulations for reducing bacteria are introduced, including applying various formulations for reducing bacteria to the oral cavity, the nasal cavity, and/or the epidermis of a user or an animal. The formulations for removing and/or reducing bacteria are applied to the oral cavity via delivery devices, including, but not limited to, a toothbrush, aerosol can, roll-on bottle, bar, and bottle, and the oral formulations for reducing bacteria can take various forms, including, but not limited to, a liquid, pellet, powder or granule, cream, spray or stick. Formulations for reducing bacteria can be applied to the epidermis of a user for removal and/or reduction of bacteria by use of a delivery device including, but not limited to, a toothbrush, aerosol can, roll-on bottle, bar, and bottle, and the epidermal formulations for reducing bacteria can take various forms, including, but not limited to a spray, roll-on, gel, solid, liquid, cream, or stick.

### TECHNICAL FIELD

Embodiments of the present invention relate generally to preventing oral and epidermal malodor in humans and animals through stopping, removing, killing, or decreasing accumulation of bacteria, fungi, and/or microbes, particularly in parts of the body that are more open to bacterial invasion (natural epidermal bacterial flora), such as the mouth, armpit, foot, and the sweaty parts of the epidermis. This is done through using formulations for reducing bacteria.

### BACKGROUND

Throughout history, humans attempted to reduce offensive odors caused by accumulation of bacteria in the oral cavity and on the epidermis.

The phenomena of bad breath or halitosis, which is a term consisting of the Latin word “Halitus” meaning “breath” and the Greek suffix “osis” meaning “unnatural state,” is an offensive odor or malodor in the oral cavity. The most common reason behind bad breath or “halitosis” in humans or animals not having systemic disorders is the increase in accumulation of oral natural bacterial flora.

Halitosis is mainly caused by the increase in accumulation and metabolism of natural bacterial flora in the oral activity and on the tongue that produce various malodorous compounds. The posterior dorsum of the tongue, when poorly cleansed, provides an ideal habitat for bacteria, so the food debris, remains of dead overlaying cells (epithelial cells), postnasal drips, and dead bacteria are favorable conditions for rapid growth and aggregation of bacteria. The accumulation and metabolism of oral bacteria, which are mostly anaerobic, produce hydrogen sulfur, with an offensive odor or malodor similar to the odor of rotten egg. The accumulation and metabolism of oral bacteria also produce other malodorous compounds such as: methyl mercaptan and skatole, which also exist in feces; indole, which is used in perfume production in small amounts but has an offensive odor in appreciable amounts; cadverine, which exists in putrefied corpses; putrescine, which is found in spoiled meat; and isovaleric acid, which has an odor like sweaty foot odor.

Halitosis is secondarily caused by the accumulation of bacterial colonies resulting from diseases and infections of the oral cavity, teeth, and gums, or through systemic illnesses. Until the present, the common ways for prevention or reduction of halitosis have been treating such diseases and infections and brushing, using toothpaste and/or an oral rinse, such as mouthwash.

Toothpastes consisting of abrasives, humectants, foaming and cleansing agents, binders, colorings, sweetener, essences, emulsifiers, preservatives, antibacterial agents, and water, are used in conjunction with a toothbrush to remove the bacterial plaque and food particles from the surface of teeth, gums, and the tongue.

Oral rinses or mouthwashes are chemotherapeutic agents used as means to kill and/or remove the bacteria that cause halitosis. The mouthwash is swished in the mouth or gargled, causing the liquid, which contains various antibacterials, to kill the bacteria, and the force of the movement of the liquid removes the bacterial coating.

Common commercial brands of mouthwash have different ingredients. The active ingredients may include alcohol, chlorhexidine digluconate, cetylpyridinium chloride, hexetidine, benzoic acid (which acts as a buffer), methyl salicylate, benzalkonium chloride, methyl paraben, hydrogen peroxide, domiphen bromide, and sometimes fluoride, enzymes, and calcium. Mouthwash can also contain essential oils that have some antibacterial properties, like phenol, thymol, eugenol, eucalyptol, or menthol.

Epidermal malodor is mainly caused by colonization of the natural epidermal bacterial flora and their metabolic activities, as well as the enzymes produced by these bacteria in sweaty parts of the body. The apocrine sweat glands produce the largest amount of sweat. The apocrine sweat glands also secrete the majority of chemical compounds needed for the natural epidermal bacterial flora to metabolize the chemical compounds into malodorous substances. This action usually happens in most regions of the body, particularly in genital regions, as well as the points at which the arms and legs are connected to the trunk (i.e. armpits, crotch), although the apocrine sweat glands can also be found in the areola, the anogenital region, and around the navel. The springy hairs of the armpits and the genital region can help to diffuse the malodors.

Epidermal malodor is influenced by the action of reproduction, accumulation, and metabolism of the natural epidermal bacterial flora, including species of Corynebacterium, which manufacture enzymes called lipases. Lipases break down the lipids in sweat to create smaller molecules like butyric acid. These smaller molecules cause the body to have its particular odor. Propionic acid (also known as “propanoic acid”) is present in many sweat samples. This acid is a degradation product of some amino acids by propionibacteria, which thrive in the ducts of adolescent and adult sebaceous glands. Because propionic acid is chemically similar to acetic acid, with similar characteristics, including odor, the body odor of some individuals may be vinegar-like. Isovaleric acid (i.e., 3-methylbutanoic acid) is the other source of body odor, and isovaleric acid is produced as a result of the activity of Staphylococcus epidermidis bacteria.

To prevent and reduce epidermal malodor, the skin is washed with soap and water. Other means for reducing epidermal malodor are to use deodorants or antiperspirants. Deodorants and antiperspirants are substances, which, by application to the body to reduce sweat secretion, affect the body odor caused by bacterial growth in armpits, feet, and other areas of the body. These are common methods that are used at present.

Likewise, perfumes and colognes are also used to mask epidermal malodor, but trying to mask the malodor does not prevent or reduce the underlying bacteria causing that malodor. The problems with the commonly known methods for prevention, treatment, or reduction of oral and epidermal malodor are that: (1) the antibacterial agents are limited; and (2) the effects of the antibacterial agents are not fast enough and do not directly target the causes of the malodor, meaning bacteria. So it is necessary to provide formulations for prevention, treatment, or reduction of oral and epidermal malodor that also have direct and fast effects on the causes of the malodors, the causes of the malodors including onion particles, water, fat, and/or dead cells. In addition, such formulations for prevention or reduction of oral and epidermal malodor have antibacterial properties.

The aforementioned and other shortcomings are illustrative rather than exhaustive. Other conventional approaches may have yet other drawbacks.

### SUMMARY OF THE INVENTION

An aspect of the present invention provides methods of treating, preventing, or reducing malodor in an oral cavity. These methods include using formulations for reducing bacteria. The formulations for reducing bacteria remove bacteria from the oral cavity of the user and reduce the accumulation of bacteria there. An aspect of the present invention provides devices used for delivering the formulations for reducing bacteria. The delivery devices for delivering the formulations for reducing bacteria spread the formulations for reducing bacteria onto or into various tissues, in minimal amounts, in solid or liquid forms, for example, wherein the delivery devices coat the oral cavity with the formulations for reducing bacteria as well as attract and remove particles and bacteria from the surface of the oral cavity.

Another aspect of the present invention provides methods for preventing or reducing epidermal malodor in a user. These methods include using formulations for reducing bacteria and are for external and/or epidermal use only. The formulations for reducing bacteria remove bacteria and reduce the accumulation of bacteria on the epidermis of a user. By using delivery devices for delivering external formulations for reducing bacteria, the devices coat the epidermis with the formulations for reducing bacteria, causing absorption and reduction of bacteria from the epidermis.

Another aspect of the present invention provides methods for reducing accumulation and growth of bacteria in the nasal cavity. These methods include using formulations for reducing bacteria that are for internal use in the nasal cavity. The internal formulations for reducing bacteria reduce the accumulation and growth of bacteria in the nasal cavity of the user. By using delivery devices for delivering internal formulations for reducing bacteria to the nasal cavity, the devices coat the nasal mucus membranes with the internal formulations for reducing bacteria.

This Summary of the Invention is provided in order to introduce a selection of concepts in a simplified form. The present inventions are further described below in the Detailed Description. This Summary of the Invention is neither intended to identify key features or essential features of the claimed subject matter, nor should it be used to limit the scope of the claimed subject matter. Furthermore, the claimed subject matter is not limited to implementations that solve any disadvantage noted in any part of this application.

The aforementioned and/or other features, aspects, details, utilities, and advantages of the present invention are: set forth in the detailed description that follows and/or illustrated in the accompanying drawings; inferred from the detailed description and/or illustrated in the accompanying drawings; and/or learned by practice of the present invention.

### BRIEF DESCRIPTION OF THE DRAWINGS

The present invention will be more readily understood from the detailed description of embodiments thereof, made in conjunction with the accompanying drawings, of which:

FIG. 1 illustrates a method of delivery for formulations for reducing bacteria in embodiments of the present invention in paste form from a tube;

FIG. 2 illustrates a method of delivery for formulations for reducing bacteria in embodiments of the present invention in tablet form;

FIG. 3 illustrates a method of delivery for formulations for reducing bacteria in embodiments of the present invention in strip form;

FIG. 4 illustrates a method of delivery for formulations for reducing bacteria in embodiments of the present invention in granule form or powder form in a package;

FIGS. 5A-5B illustrate a method of delivery for formulations for reducing bacteria in embodiments of the present invention in aerosol form;

FIG. 6 illustrates a method of delivery for formulations for reducing bacteria in embodiments of the present invention in liquid form.

FIG. 7 illustrates a dental floss with formulations for reducing bacteria in embodiments of the present invention, for use in the spaces between the teeth;

FIG. 8 illustrates a method of applying formulations for reducing bacteria in embodiments of the present invention in solid form with the aid of a toothbrush;

FIG. 9 illustrates the epidermis of a user receiving external formulations for reducing bacteria in embodiments of the present invention in liquid form or gel form, which are shown as methods of delivering external formulations for reducing bacteria in embodiments of the present invention for application on the epidermis;

FIG. 10 illustrates a method of applying formulations for reducing bacteria in embodiments of the present invention in paste form; and

FIG. 11 illustrates a method of application for formulations for reducing bacteria in embodiments of the present invention in stick form or pencil-like form as part of a method for delivery of formulations for reducing bacteria in embodiments of the present invention for use between toes.

### DETAILED DESCRIPTION OF THE EMBODIMENTS

Reference will now be made in detail to embodiments of the present invention, examples of which are illustrated in the accompanying drawings, wherein like reference numerals refer to the like elements throughout. The embodiments are described below to explain the present invention by referring to the figures.

Although the following text sets forth a detailed description of at least one embodiment or implementation, it is to be understood that the legal scope of protection of this application is defined by the words of the claim set forth at the end of this disclosure. The detailed description is to be construed as exemplary only and does not describe every possible embodiment. Numerous alternative embodiments and/or implementations are both contemplated and possible, using either current technology or technology developed after the filing date of this patent, which would still fall within the scope of the claims.

It is to be understood that, unless a term is expressly defined in this application using the sentence, “As used herein, the term ‘ ’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as not to confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. §112(f).

As used herein, a delivery device for delivering a solid or liquid formulation for reducing bacteria in embodiments of the present invention can include, but is not limited to, an aerosol can, bottle, roll-on bottle, and toothbrush.

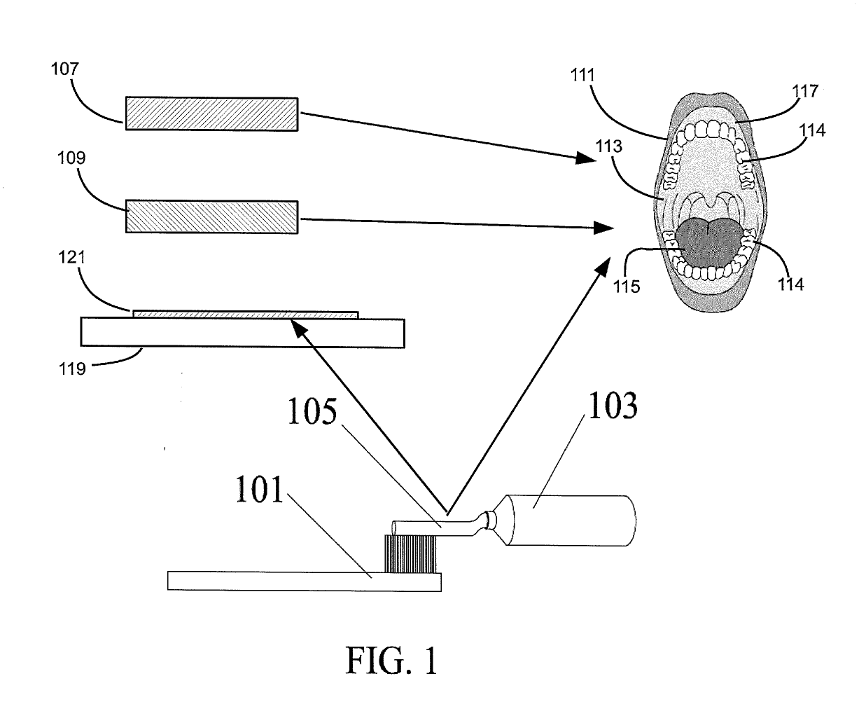

Referring now to FIG. 1, the toothbrush 101 receives the formulation for reducing bacteria in paste form 105 by application of pressure on the outer surface or distal end of the tube 103, forcing the formulation for reducing bacteria in paste form 105 out of a nozzle of the tube 103 onto the upper surface of the bristles of the toothbrush 101.

After brushing the teeth (as a physical action) with the toothpaste 107 and, then, washing the teeth 114 of an oral cavity 111 with water, the formulation for reducing bacteria in paste form 105 is applied to the teeth 114, gums 117, mouth mucus membrane (not shown), and the surface of the tongue 115 and inner cheek wall 113, especially the posterior portion of the tongue 115 using the formulation for reducing bacteria in paste form 105 via the toothbrush 101 such that all of these surfaces will be swept away and particles (not shown), dead cells (not shown), bacteria (not shown), and fats (not shown) will be brushed away by the formulation for reducing bacteria in paste form 105. The swept away debris items will leave the mouth after rinsing the mouth with water (not shown). The mouth will be cleansed and the bacterial colonies (not shown) will be removed and/or reduced thereby, and the oral malodor will be prevented or reduced, as bad breath is caused by the metabolism of colonies of natural oral bacterial flora. The bacterial membrane is formed from lipid and the formulation for reducing bacteria in paste form 105 contains soap, which is an absorbent of fat; as the result, the formulation for reducing bacteria in paste form 105 absorbs bacterial colonies and causes their reduction. The formulation for reducing bacteria in paste form 105 optionally further contains chlorhexidine digluconate, which kills the bacteria.

One possible embodiment of a formulation for reducing bacteria in paste form 105 for use in the method illustrated in FIG. 1 is provided in Table 1 below, including possible materials to be used as optional ingredients and approximate minimum and/or maximum percentages of usage of the ingredients. It will be understood that certain formulations for reducing bacteria exemplified by Formulation 1 in pellet, strip, solid, or granulated or powdered forms do not include water, and that the percentages of remaining ingredients listed below will be adjusted to total 100% by weight for such formulations for reducing bacteria in pellet, strip, powdered, or stick (pencil-like) solid forms. It will be understood that no oral/internal formulation for reducing bacteria will include zinc pyrithione. In the present disclosure, the term “internal” means that the formulation for reducing bacteria is applicable to oral cavity 111 or nasal cavity and is applied in regions such as the mouth, mucus membranes, tongue, gums, and teeth. Formulation 1 can optionally be prepared without using chlorhexidine digluconate. For users who frequently suffer from aphthosis stomatitis due to the usage of common toothpastes, and thus are sensitive to the surfactants in those toothpastes, Formulation 1 is the preferably formulation for reducing bacteria for prevention or reduction of recurrent aphthosis in such users' oral cavities. Such users can also apply other oral formulations of this invention for cleaning their oral cavities and teeth.

TABLE NO. 1
Formulation 1: Oral/Internal Formulation
(Toothpaste and Formulation for Reducing Bacteria)

| | | Approximate Minimum |
|---|---|---|
| | | and/or Maximum |
| | | Percentages of Ingredients |
| Category of Ingredient | Possible Ingredient Selection | By Weight |
| Polisher/abrasive | Silica/sodium meta phosphate | 10-50 |
| Humectant | Glycerol/Sorbital | 10-30 |
| Foaming agents/Absorption | Soap | 3-20 |
| and Reduction of Bacteria | | |
| Antibacterial | Chlorhexidine Digluconate | 0.1-0.2 |
| Adhesive/binder agent | Sodium Carboxymethyl | 0.5-3 |
| | Cellulose (“SCMC”) | |
| Sweetening agent | Sodium saccharine | 0.05-0.5 |
| Flavoring agent | Essence/menthol | 1-3 |
| Coloring agent | Titanium dioxide (TiO₂) | <1 |
| Preservative | Parabens/propyl parabens | 0.1-0.5 |
| Water | Distilled water | Remainder, up to 100% by |
| | | weight |

The formulation for reducing bacteria in paste form 105 is combined with toothpaste 107 producing combination 109 of the formulation for reducing bacteria in paste form 105 and toothpaste 107. The resulting combination 109 forms a double-purpose paste so that both the properties of the toothpaste 107 and the formulation for reducing bacteria in paste form 105 cause absorption and prevention or reduction of bacterial colonies, and prevent or reduce corresponding oral malodor, so the brushing action and prevention or reduction of oral malodor is done in one phase and its application for the user would be more convenient. Soap causes absorption and prevention or reduction of bacteria because the membranes of all bacteria consist of fat, and soap is a fat absorbent. Further, chlorhexidine digluconate kills microbes.

The formulation for reducing bacteria in paste form 105 is also applied, like a cream, to sweaty parts of human or animal epidermis so that a layer of the formulation for reducing bacteria in paste form 105 will be left on the epidermis. There is no need to wash the body after applying the formulation for reducing bacteria in paste form 105 to the epidermis. As such, it is advisable to apply the formulation for reducing bacteria in paste form 105 after taking a bath or washing the epidermis.

Additionally, the formulation for reducing bacteria in paste form 105 attracts fats and existing water in the sweat and on the epidermis 119. As a result, in the absence of fat and water removed in the presence of the formulation for reducing bacteria in paste form 105, which contains soap, and optionally chlorhexidine digluconate, and further optionally zinc pyrithione, the colonies of natural bacterial flora of epidermis 119 will be absorbed by the soap, because the membranes of all bacteria consist of fat, and soap is a fat absorbent. Further, chlorhexidine digluconate and zinc pyrithione kill microbes and prevent or reduce epidermal malodor.

At present, for the formulation for reducing bacteria in paste form 105, soaps with a neutral pH or pH about 7, or weakly acidic soaps (with a pH of about 6.5) or weakly alkaline (with a pH of about 7.5), and preferably a pH of about 7 are recommended.

It will be understood that the means of apply the formulation for reducing bacteria in paste form 105 also has application in animals.

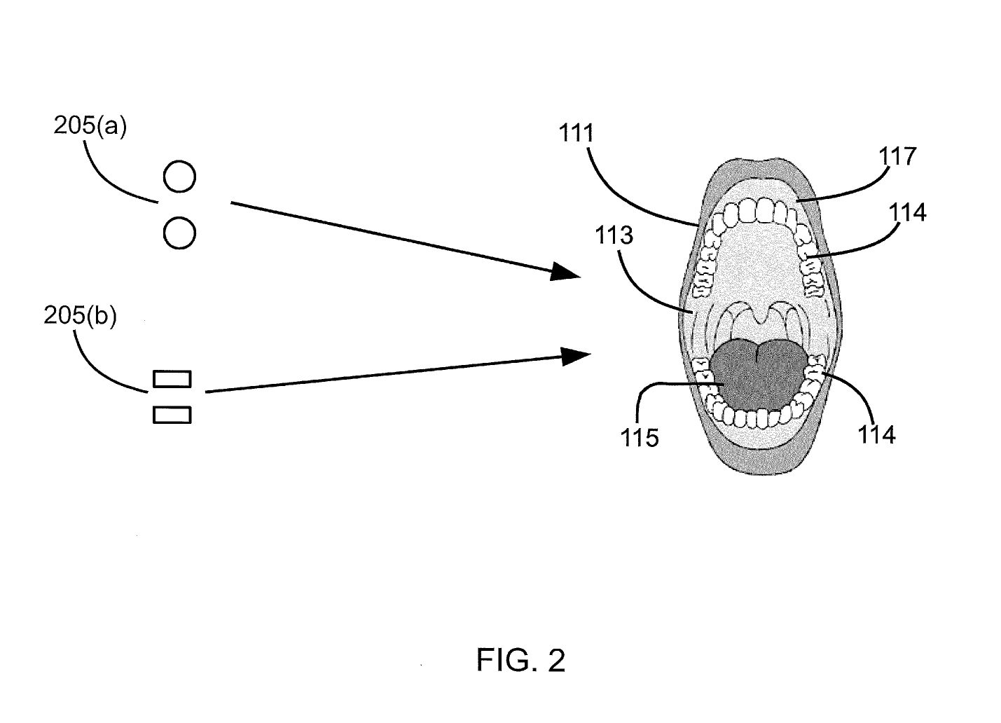

Referring now to FIG. 2, a formulation for reducing bacteria in pellet form, which is shown in top view 205(a) and side view 205(b), is illustrated. The formulation for reducing bacteria in pellet form 205(a-b) can be stored in a box (not shown) or other packaging.

The formulation for reducing bacteria in pellet form 205(a-b) is used in situations where the user has no access to water and therefore cannot rinse the oral cavity 111. The formulation for reducing bacteria in pellet form 205(a-b) is especially useful in low-water environments such as deserts and mountainsides. The formulation for reducing bacteria in pellet form 205(a-b) is placed in the oral cavity 111, especially on the surface of the tongue 115. Saliva generated by the oral cavity 111 will mix with the formulation for reducing bacteria in pellet form 205(a-b). Via movement of the saliva and the formulation for reducing bacteria in pellet form 205(a-b), the inner cheek walls 113, the teeth 114, tongue 115, and gums 117 will be coated with the amalgamation formed by the saliva and the formulation for reducing bacteria in pellet form 205(a-b).

The amalgamation of the saliva and the formulation for reducing bacteria in pellet form 205(a-b) will be spit out, removing bacteria (not shown) and particles (not shown). The result is prevention or reduction of the bacterial colonies (not shown) in the oral cavity 111 and removing and/or reducing the related oral malodor caused by them. Preferably, the formulation for reducing bacteria in pellet form 205(a-b) will have a pH of about 7 because the person who consumes the formulation for reducing bacteria in pellet form 205(a-b) has no access to water.

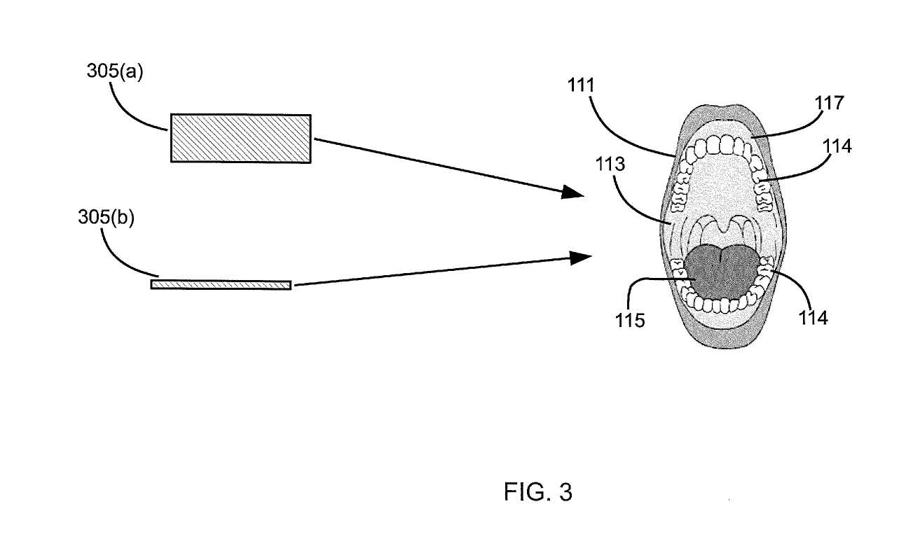

Referring now to FIG. 3, a formulation for reducing bacteria in strip form is illustrated in top view 305(a) and side view 305(b). The formulation for reducing bacteria in strip form 305(a-b) can be stored in a flip-top box (not shown) or other packaging.

The formulation for reducing bacteria in strip form 305(a-b) is used in situations in which the user has no access to water and therefore cannot rinse the oral cavity 111. The formulation for reducing bacteria in strip form 305(a-b) is especially useful in low-water environments such as deserts and mountainsides. The formulation for reducing bacteria in strip form 305(a-b) can be removed from its packaging and placed in the oral cavity 111, especially on the surface of the tongue 115. Saliva generated by the oral cavity 111 will mix with the formulation for reducing bacteria in strip form 305(a-b). Via movement of the saliva and the formulation for reducing bacteria in strip form 305(a-b), the inner cheek walls 113, the teeth 114, tongue 115, and gums 117 will be coated with the amalgamation of the saliva and the formulation for reducing bacteria in strip form 305(a-b).

The amalgamation of the saliva and the formulation for reducing bacteria in strip form 305(a-b) will be spit out, removing bacteria (not shown) and particles (not shown). The result is that the bacterial colonies (not shown) in the oral cavity 111 will be removed and/or reduced, and oral malodor will be prevented or reduced. Preferably, the formulation for reducing bacteria in strip form 305(a-b) will have a pH of about 7 because the person who consumes it has no access to water.

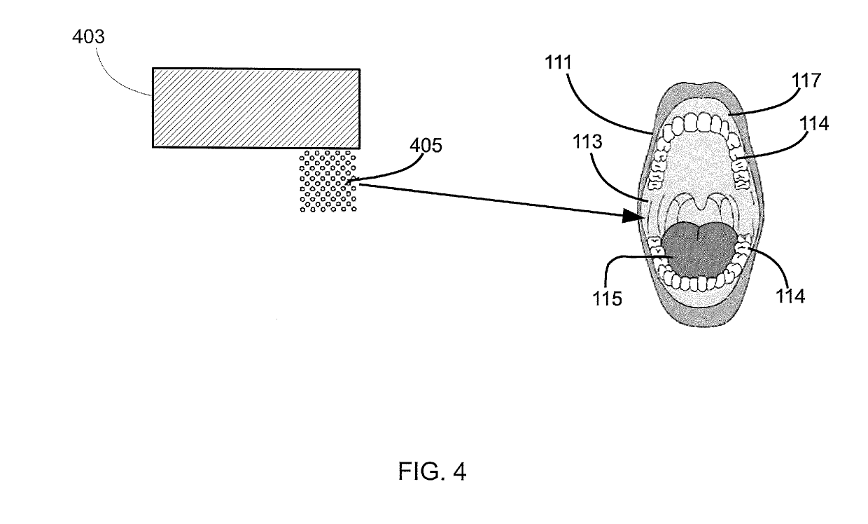

Referring now to FIG. 4, a formulation for reducing bacteria in granulated or powdered form 405 stored in a paper packet 403 is illustrated. The formulation for reducing bacteria in granulated or powdered form 405 is used in situations in which the user has no access to water and therefore cannot rinse the oral cavity 111. The formulation for reducing bacteria in granulated or powdered form 405 is especially useful in low-water environments such as deserts and mountainsides. The formulation for reducing bacteria in granulated or powdered form 405 can be removed from its package 403 and placed in the oral cavity 111, especially on the surface of the tongue 115. Saliva generated by the oral cavity 111 will mix the formulation for reducing bacteria in granulated or powdered form 405. Via movement of the saliva and the formulation for reducing bacteria in granulated or powdered form 405, the inner cheek walls 113, the teeth 114, tongue 115, and gums 117 will be coated with the amalgamation of the saliva and the formulation for reducing bacteria in granulated or powdered form 405.

The amalgamation of the saliva and the formulation for reducing bacteria in granulated or powdered form 405 will be spit out, removing bacteria (not shown) and particles (not shown). The result is that the bacterial colonies (not shown) in the oral cavity 111 will be prevented or reduced, and oral malodor will be removed and/or reduced. Preferably, the formulation for reducing bacteria in granulated or powdered form 405 will have a pH of about 7 because the person who consumes it has no access to water.

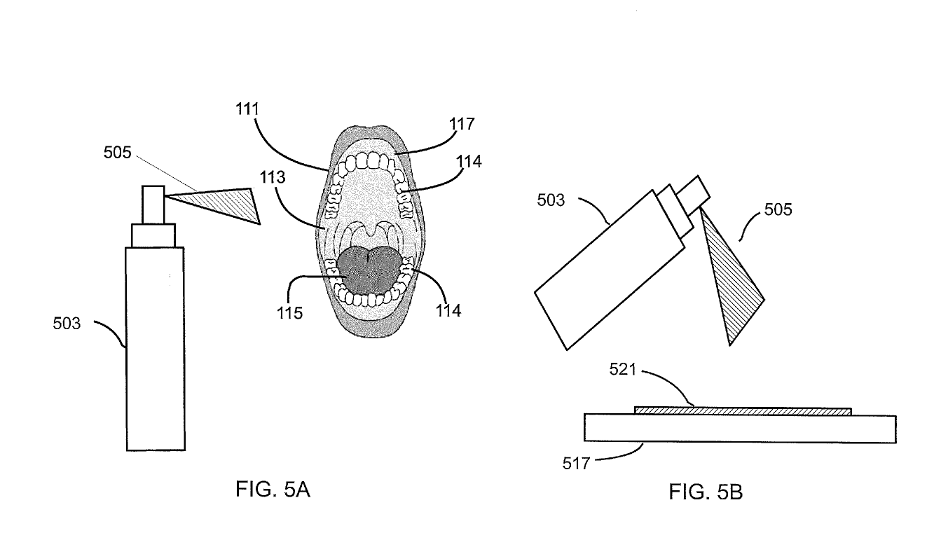

Referring now to FIGS. 5A-5B, a user who is receiving a formulation for reducing bacteria in aerosol form 505 from the spray can 503 is illustrated. In FIG. 5A the formulation for reducing bacteria in aerosol form 505 is applied to the oral cavity 111 of a user (human or animal). After brushing the teeth 114 with toothbrush 101 using toothpaste 107, in the manner described above with regards to FIG. 1, the formulation for reducing bacteria in aerosol form 505 is applied to the oral cavity 111 by spraying from spray can 503. Then, the formulation for reducing bacteria in aerosol form 505 will be rinsed away with water, preventing or reducing bacterial colonies in the oral cavity 111, thereby preventing or reducing the bacteria that causes oral malodor. If the formulation for reducing bacteria in aerosol form 505 is not rinsed away and only spit out from the mouth, bacterial colonies will be removed and/or reduced, reducing oral malodor. Further, the formulation for reducing bacteria in aerosol form 505 causes prevention or reduction of infection in the oral cavity 111b.

Referring now to FIG. 5B, the formulation for reducing bacteria in aerosol form 505 is sprayed on the epidermis 517 of a human or animal from the spray can 503. It would be understood that the epidermis 517 would be those areas of a human or animal that are prone to the production of sweat and bacterial colonies that cause epidermal malodor. Such areas could be the armpits, feet, or other areas of the epidermis 517 of a human or animal. Preferably, the formulation for reducing bacteria in aerosol form 505 is applied after taking a bath or washing the epidermis 517 to form coating 521, which remains on the epidermis 517.

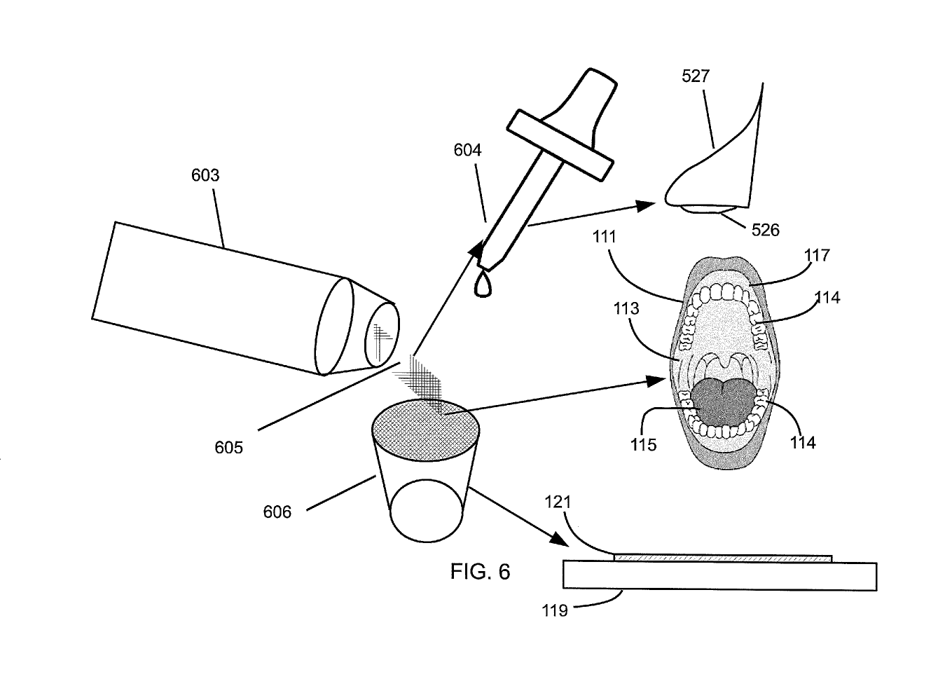

Referring to FIG. 6, a formulation for reducing bacteria in liquid form 605 is applied to the oral cavity 111, epidermis 119, or nasal cavity 526 of a nose 527 as an antiseptic wash. The formulation for reducing bacteria in liquid form 605 is stored in the bottle 603 and poured into a cup 606. It is better that, after brushing the teeth 114 by using a toothbrush 101 and toothpaste 107 in the manner discussed in FIG. 1, the user pours the formulation for reducing bacteria in liquid form 605 from the cup 606 into the oral cavity 111. The user gargles the formulation for reducing bacteria in liquid form 605 and spits the formulation for reducing bacteria in liquid form 605 out, removing and/or reducing bacterial colonies (not shown), dead cells (not shown), and other particles (not shown) from the oral cavity 111, thereby preventing or reducing the cause of oral malodor. The user can further rinse the oral cavity 111 with water (not shown) to remove the formulation for reducing bacteria in liquid form 605 and any residual bacteria (not shown), dead cells (not shown), and other particles (not shown) from the oral cavity 111.

The formulation for reducing bacteria in liquid form 605 can be removed from the bottle 603 or cup 606 using a dropper 604. The dropper 604 is used to drop the formulation for reducing bacteria in liquid form 605 into the nasal cavity 526 of the nose 527. It should be understood that the formulation for reducing bacteria in liquid form 605 can also be introduced to the nasal cavity 526 by inhaling from the user's clean hand.

After introducing the formulation for reducing bacteria in liquid form 605, the nasal cavity 526 is rinsed using water (not shown). It will be understood that saline should not be used to rinse the formulation for reducing bacteria in liquid form 605 from the nasal cavity 526 and/or sinuses in order to prevent the mucus membrane from drying out.

The formulation for reducing bacteria in liquid form 605 may also be applied to prevent or reduce bacterial colonies from the surface of the epidermis 119 in the form of a coating 121. Preferably, the formulation for reducing bacteria in liquid form 605 will be applied to the epidermis 119 after taking a bath or washing.

The formulation for reducing bacteria in liquid form 605 preferably has a neutral pH (of about 7). This pH of about 7 is preferred for washing the nose and sinuses.

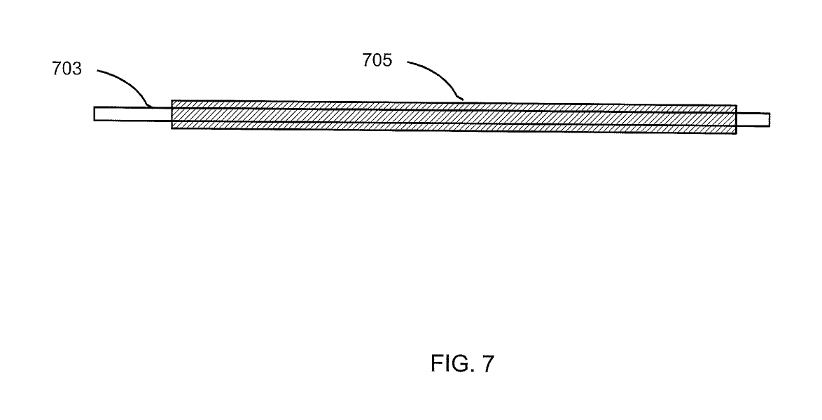

Referring now to FIG. 7, a section of dental floss 703 is illustrated. The dental floss 703 is covered with a formulation for reducing bacteria in solid form 705. The dental floss 703 is used in the conventional manner to floss between the teeth 114. The application of dental floss 703 containing the formulation for reducing bacteria in solid form 705 between the teeth 114 performs the dual purpose of removing particles (not shown) and removing and/or reducing bacterial colonies between the teeth 114, thereby causing the prevention or reduction of oral malodor between teeth.

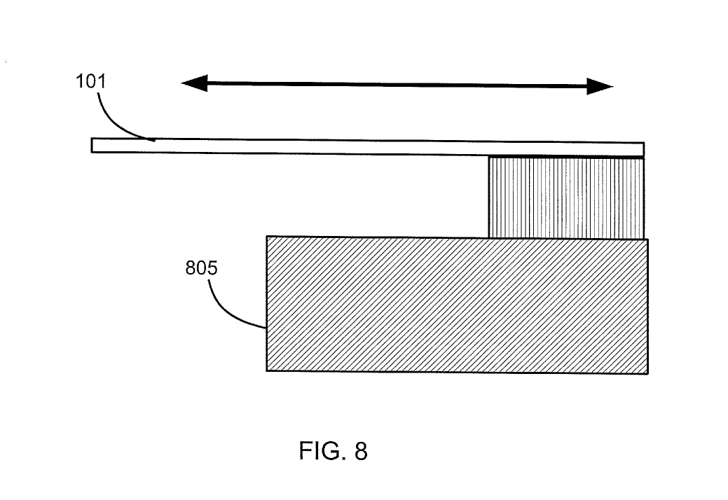

Referring now to FIG. 8, a formulation for reducing bacteria in solid form 805 is illustrated. The toothbrush 101 is moistened with water (not shown) and then rubbed across the surface of the formulation for reducing bacteria in solid form 805. After brushing the teeth 114 with toothbrush 101 using toothpaste 107 in the manner described above with regards to FIG. 1, the formulation for reducing bacteria in solid form 805 is applied to the oral cavity 111 in the same manner discussed above with regards to FIG. 1.

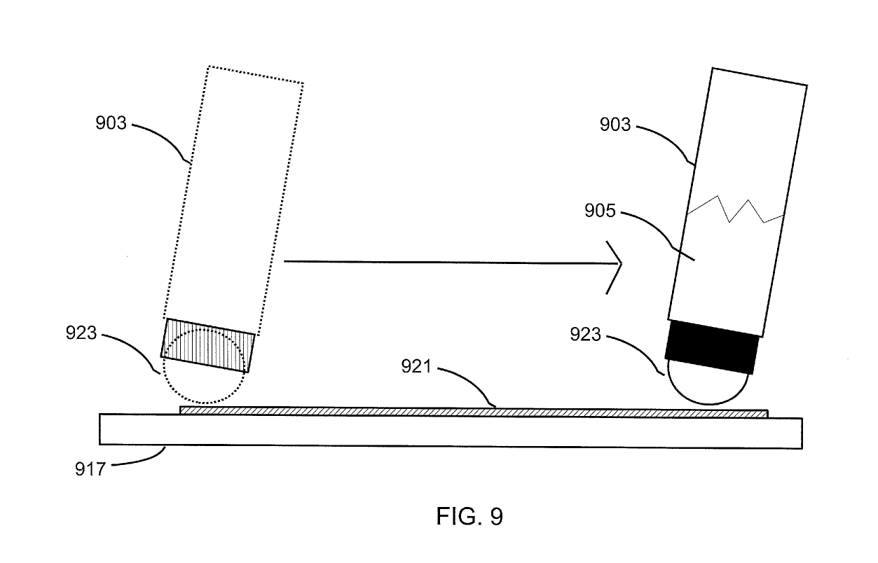

Referring now to FIG. 9, a formulation for reducing bacteria in gel form 905 from a roll-on bottle 903 is illustrated. The bottle 903 contains the formulation for reducing bacteria in gel form 905, which is applied via a roll-on applicator 923 moving across the epidermis 917, leaving residue 921 of the formulation for reducing bacteria in gel form 905 on the epidermis 917. While a roll-on applicator 923 is illustrated, the applicator may also be a sponge or brush or other applicator of liquids, known in the art.

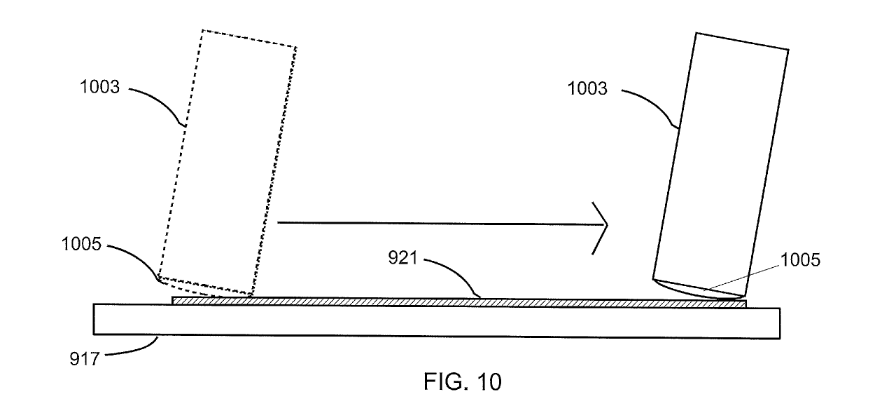

Referring now to FIG. 10, an external formulation for reducing bacteria in paste form 1005 from a dispenser 1003 is illustrated. Optionally, the external formulation for reducing bacteria in paste form 1005 can be a dispenser 1003 covering the external formulation for reducing bacteria in paste form 1005. The external formulation for reducing bacteria in paste form 1005 is applied to the epidermis 917 several times, after washing the epidermis 917 with soap and water and drying with a towel (not shown). The surface of the epidermis 917 then receives the external formulation for reducing bacteria in paste form 1005, thereby leaving a coating 921 of the formulation for reducing bacteria in paste form 1005 on the epidermis 917.

The coating 921 of the external formulation for reducing bacteria in paste form 1005 on the epidermis 917 thereby causes prevention or reduction of bacterial colonies on the epidermis 917. There is no need to wash the epidermis 917 after applying the external formulation for reducing bacteria in paste form 1005 to the epidermis 917. It is better to apply the external formulation for reducing bacteria in paste form 1005 after taking a bath or washing the skin.

Additionally, the external formulation for reducing bacteria in paste form 1005 attracts the fats and water in sweat and on the epidermis 917, and absorbs natural bacterial flora colonies on the epidermis 917, which will reduce epidermal malodor.

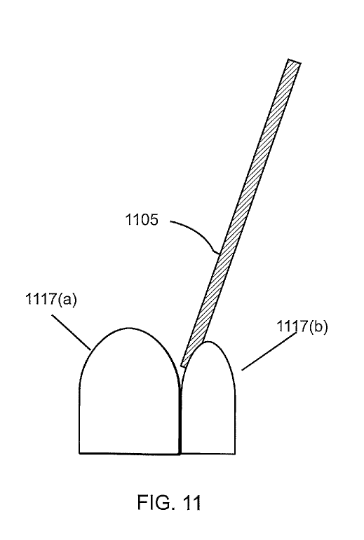

Referring now to FIG. 11, application of a formulation for reducing bacteria in solid form 1105, applied to the areas between the toes 1117(a) and 1117(b), is shown. The formulation for reducing bacteria in solid form 1105 has a stick or pencil-like form. Optionally, the formulation for reducing bacteria in solid form 1105, which has a stick or pencil-like form, can be applied directly to the areas between the toes 1117(a) and 1117(b), or the formulation for reducing bacteria in solid form 1105, which has a stick or pencil-like form, could be moistened with water and rubbed over the areas between the toes 1117(a) and 1117(b) so that a layer of the formulation for reducing bacteria in solid form 1105 will remain on the areas between the toes 1117(a) and 1117(b).

Alternatively, the formulation for reducing bacteria in solid form 1105, which has a stick or pencil-like form, may be applied to the oral cavity 111 by sucking on it in the manner one sucks on candy. The user can then spit out the formulation for reducing bacteria in solid form 1105 mixed with saliva, reducing and/or removing bacterial colonies (not shown), dead cells (not shown), and other particles (not shown) from the oral cavity 111, thereby reducing the causes of oral malodor. The user can further rinse out the oral cavity 111 with water (not shown) to remove the formulation for reducing bacteria in solid form 1105 and any residual of bacteria (not shown), dead cells (not shown), and other particles (not shown) from the oral cavity 111. The formulation for reducing bacteria in solid form 1105, which has a stick or pencil-like form, if applied to the oral cavity 111, must not contain zinc pyrithione.

In addition to Formulation No. 1, ingredients comprising alternative embodiments of formulations for reducing bacteria are shown in Table No. 2. It will be understood that formulations for reducing bacteria that are illustrated by the above embodiments may be one of the following formulations, with the understanding that formulations including zinc pyrithione are specifically for external use only, applicable according to the suggested methods that follow.

TABLE NO. 2
Formulations 2-13

| | | | | % | % | | % |
|---|---|---|---|---|---|---|---|
| Formu- | | Place of | Total % | Soap/ | Chlorhexidine | % Zinc | Distilled |
| lation # | Form | Use | by Weight | Powder | digluconate | pyrithione | water |
| 2 | Liquid | Oral or | 100 | 5 | 0.1 | — | 94.9 |
| | or spray | epidermal | | | | | |
| 3 | Liquid | Oral or | 100 | 5-25 | — | — | 75-95 |
| | or spray | epidermal | | | | | |
| 4 | Liquid | Epidermal | 100 | 5-25 | 0.05-0.2 | 0.05-0.2 | 72.8-94.9 |
| | or spray | | | | | | |
| 5 | Cream/ | Oral or | 100 | 40 | — | — | 60 |
| | Gel/Roll | epidermal | | | | | |
| 6 | Cream/ | Oral or | 100 | 20-40 | 0.05-0.2 | — | 59.8-9.95 |
| | Gel/Roll | epidermal | | | | | |
| 7 | Cream/ | Epidermal | 100 | 15-40 | 0.05-0.2 | 0.25-2 | 57.8-84.7 |
| | Gel/Roll | | | | | | |
| 8 | Cream/ | Epidermal | 100 | 15-35 | 0.05-0.2 | 0.25-2 | 62.8-84.7 |
| | Gel/Roll | | | | | | |
| 9 | Solid, pellet, | Oral or | 100 | 100 | — | — | — |
| | strip, granule | epidermal | | | | | |
| | or powder, | | | | | | |
| | stick | | | | | | |
| 10 | Solid, pellet, | Oral or | 100 | 99.8-99.95 | 0.05-0.2 | — | — |
| | strip, granule | epidermal | | | | | |
| | or powder, | | | | | | |
| | stick | | | | | | |
| 11 | Solid/stick | Epidermal | 100 | 97.8-99.7 | 0.05-0.2 | 0.25-2 | — |
| 12 | Solid, pellet, | Epidermal | 100 | 99.5 | — | 0.5 | — |
| | strip, granule | | | | | | |
| | or powder, | | | | | | |
| | stick | | | | | | |
| 13 | Cream | Epidermal | 100 | 30 | — | 0.25 | 69.75 |

As described above, the present invention applies soap in a new and novel manner in order to reduce bacterial accumulation and the associated malodor caused by bacterial metabolism.

The soaps are formed from oils or fats like glyceryl ester acids, preferably, Palmitin, Stearin, and Olein, which are found in pig fat, whale fat (animal fats), olive oil, coconut oil, and flax seed oil (vegetable oils). The soaps are produced out of the reaction of any kind of oil or fat with sodium hydroxide or potassium hydroxide, and the produced glycerin remains in soaps as a by-product resulting from the soap-producing reaction (glycerin is skin softener).

One or more suitable saponification methods are contemplated here. In one exemplary reaction, distilled water is used for dissolving sodium hydroxide or potassium hydroxide, and the liquid is stirred as the temperature increases gradually until all of the sodium hydroxide or potassium hydroxide is dissolved in the water. The liquid should then be cooled down to reach a temperature of about 45° C. Solid oil or animal fat is separately warmed in warm water, or heated with steam, to reach a temperature of about 45° C. The aqueous sodium hydroxide or potassium hydroxide is then slowly added to the oils and the mixture is stirred to advance the reaction and make the liquid more viscous. The oil or fat reacts with the sodium hydroxide or potassium hydroxide, and the generated heat evaporates the water, causing the soap to gradually solidify. Deodorants and extracts are used to give a good smell to soaps and to give a better appearance to them, or edible pigments are used and may be so added to soaps that are used as the cleansing product of the present invention.

Preparative protocols for two specific formulations follow as examples:

Example A: Oral formulation for reducing bacteria in cream form; and

Example B: Epidermal formulation for reducing bacteria in cream form.

The formulations for reducing bacteria, used for reducing oral and/or epidermal malodor, can contain ingredients including, but not limited to: soap, chlorhexidine digluconate, and distilled water. Only epidermal formulations for reducing bacteria of the present invention may include zinc pyrithione.

### Example A: Oral Formulation for Reducing Bacteria in Cream Form

Ingredients comprising the oral formulation for reducing bacteria in cream form of Example A are shown in Table No. 3.

TABLE NO. 3

| Total % by | % of Soap/Powder | % chlorhexidine | % distilled |
|---|---|---|---|
| Weight | (with pH about 6.5-8.5) | digluconate | water |
| 100 | 20-40 | 0.05-0.2 | 59.8-79.95 |

(Percentages are by weight and based on total weight of the formulation.)

Field of Action:

Soap is a hydrophobic compound that is fat absorbent. Soap absorbs fat existing in the oral cavity, as well as bacteria and bacterial colonies of natural oral bacterial flora, because the membranes of all bacteria are formed from lipids. Soap also has anti-surface adhesion properties and can cause surface tension reduction in particles, resulting in the separation of malodorous food particles (such as onion particles), bacteria, dead cells, and bacterial plaque from oral membrane surfaces, the teeth, and the tongue.

Chlorhexidine, e.g. as digluconate salt, due to its high cationic properties, binds to the anionic groups existing on the mouth mucus membrane surface. Chlorhexidine will gradually and slowly release after its adhesion, and until its release chlorhexidine will maintain an antimicrobial environment in the oral cavity. Approximately thirty percent of the total consumed amount of chlorhexidine will remain in the oral cavity.

Chlorhexidine is effective on a wide range of gram-positive and gram-negative bacteria, as well as some fungi and viruses, such as viruses causing HIV and hepatitis. Due to the cationic properties of chlorhexidine, the absorption of chlorhexidine through the digestive system is minimal, and current animal research does not demonstrate any evidence of toxic and/or cancerous effects in animals due to chlorhexidine exposure. Chlorhexidine has a high affinity for adhesion, and strong binding to the bacterial membrane. After attachment of chlorhexidine to the bacterial membrane, the penetration of the membrane will be increased. Due to the disruption of the osmotic equilibrium in the two sides of the bacterial membrane, some of the contents inside the bacteria with low molecular weight will permeate through the membrane out of the bacteria. This phenomenon occurs in the presence of small amounts of chlorhexidine, while higher amounts of chlorhexidine result in sediment in the bacterial cytoplasm. In addition, due to its cationic properties, chlorhexidine controls the glycolysis action in bacteria.

Conclusion:

The oral formulation for reducing bacteria in cream form reduces and removes factors causing oral malodor, and disinfects the oral cavity, by causing intensive and fast reduction of bacteria and bacterial colonies of the natural oral bacterial flora. This results in prevention, treatment, or reduction of a human's or animal's oral malodor, and delays recurrence of oral malodor for an appreciable time period.

Considering the limited side effects of chlorhexidine and soap, which do not include any toxic or systemic complications, and the considerable antimicrobial effects of chlorhexidine and soap, these materials are recommended for reduction of oral malodor in this embodiment so that humans and animals can both enjoy the extensive advantages of these two substances.

Method of Application:

The method of using the Example A oral formulation for reducing bacteria in cream form along with a toothbrush is similar to the common application method of using a toothbrush with toothpaste in an oral cavity. Referring to FIG. 1, the user will put approximately one centimeter of oral formulation for reducing bacteria in cream form from the tube on a toothbrush 101 and brush the teeth 114 and tongue 115. Then the user will wash and rinse the oral cavity 111 with water. The user can brush the oral cavity 111 twice daily (i.e., morning and night) with the cream.

Note:

Optionally, approved additives such as colors, scents, essences, flavoring agents, preservatives, or softeners can be added to all of the oral and epidermal formulations for reducing bacteria in embodiments of the present invention. Also, for use by animals, approved additives can be added to the formulations for reducing bacteria in embodiments of the present invention. For example, approved meat flavoring can be used as an approved additive for dogs. It is also possible to prepare toothpaste for animals by adding oral formulations for reducing bacteria from Table No. 2 to children's edible toothpastes.

It is expected that the comparison of the oral cavity of a user of an oral formulation for reducing bacteria in cream form of the present invention (Table No. 3), under the experiment, will show that according to the bacterial count, measured by Colony—Forming Unit (“CFU”), intensive removal and/or reduction of bacteria and bacterial colonies in saliva occur after use of the oral formulation for reducing bacteria in cream form (Table No. 3) and thus cause prevention or reduction of oral malodor.

### Example B: Epidermal Formulation for Reducing Bacteria in Cream Form

Ingredients comprising the oral formulation for reducing bacteria in cream form of Example B are shown in Table No. 4.

TABLE NO. 4

| Total % | | % | | |
|---|---|---|---|---|
| by | % of Hard Soap (with | chlorhexidine | % zinc | % distilled |
| Weight | pH about 6.5-8.5) | digluconate | pyrithione | water |
| 100 | 15-40 | 0.05-0.2 | 0.25-2 | 57.8-84.7 |

(Percentages are by weight and based on total weight of the formulation.)

Field of Action:

Soap is a hydrophobic compound that is fat absorbent. Soap absorbs fat existing on the epidermis, as well as bacteria and bacterial colonies of natural epidermal bacterial flora, because the membranes of all bacteria are formed from lipids. Soap also has anti-surface adhesion properties and can cause surface tension reduction in particles, resulting in the separation of malodorous particles, bacteria, dead cells, and bacterial plaque from the epidermis.

Chlorhexidine, e.g. as digluconate salt, due to its high cationic properties, binds to the anionic groups existing on the epidermis membrane surface. Chlorhexidine will gradually and slowly release after its adhesion, and until its release chlorhexidine will maintain an antimicrobial environment on the epidermis. Approximately thirty percent of the total consumed amount of chlorhexidine will remain in the oral cavity.

Chlorhexidine is effective on a wide range of gram-positive and gram-negative bacteria, as well as some fungi and viruses, such as viruses causing HIV and hepatitis. Due to the cationic properties of chlorhexidine, the absorption of chlorhexidine through the digestive system is minimal, and current animal research does not demonstrate any evidence of toxic and/or cancerous effects in animals due to chlorhexidine exposure. Chlorhexidine has a high affinity for adhesion, and strong binding to the bacterial membrane. After attachment of chlorhexidine to the bacterial membrane, the penetration of the membrane will be increased. Due to the disruption of the osmotic equilibrium in the two sides of the bacterial membrane, some of the contents inside the bacteria with low molecular weight will permeate through the membrane out of the bacteria. This phenomenon occurs in the presence of small amounts of chlorhexidine, while higher amounts of chlorhexidine result in sediment in the bacterial cytoplasm. In addition, due to its cationic properties, chlorhexidine controls the glycolysis action in bacteria.

Zinc pyrithione, with its anti-fungal, antibacterial, and anti-inflammatory properties, controls the growth of gram-positive bacteria, gram-negative bacteria, yeast, mold, streptococcus pathogens, staphylococcus, and athlete's foot fungi. Also, by regulating the secretion of sebaceous glands, zinc pyrithione acts as an anti-seborrheic, and with its anti-mitotic effects, causes the reduction of the formation of epithelial cells (epidermal).

Conclusion:

The epidermal formulation for reducing bacteria in cream form removes and/or reduces factors causing epidermal malodor, and disinfects the epidermis, by causing intensive and fast removal and.or reduction of bacteria and bacterial colonies of the natural epidermal bacterial flora. This results in prevention or reduction of a human's or animal's epidermal malodor, and delays recurrence of epidermal malodor for an appreciable time period.

Notes:

Upon the observation of any complication and/or allergy, usage of the formulation for reducing bacteria must be discontinued. There must be no contact made between any formulation for reducing bacteria and a user's eyes. Further, the formulations for reducing bacteria must not be applied near the ears. Formulations for reducing bacteria that contain zinc pyrithione must not be applied in the nasal cavity of the user nor swallowed. For dry and sensitive skin, it is preferred to use baby soap with a neutral pH of 7 and humectants like glycerin in the amount of approximately 10-30 percent in formulation of the cream.

Method of Application:

It is better to apply the external formulation for reducing bacteria in cream form on the epidermis after a bath to benefit its maximum antibacterial strength. The external formulation for reducing bacteria in cream form should be applied once or twice daily (i.e., morning and/or evening) on the epidermis surface, under the armpits, between the toes, and on the other perspiring regions of the epidermis.

It is expected that the comparison of the epidermis of a user before and after applying the external formulation for reducing bacteria in cream form of the present invention (Table No. 4) on the epidermis of the user, under the experiment, will show that according to the bacterial count, measured by Colony—Forming Unit (“CFU”), intensive removal and/or reduction of bacteria and bacterial colonies on the epidermis occur after use of the external formulation for reducing bacteria in cream form (Table No. 4) and thus cause prevention or reduction of external malodor.

Examples of various features/aspects/components/operations have been provided to facilitate understanding of the disclosed embodiments of the present invention. In addition, various preferences have been discussed to facilitate understanding of the disclosed embodiments of the present invention. It is to be understood that all examples and preferences disclosed herein are intended to be non-limiting.

Although selected embodiments of the present invention have been shown and described individually, it is to be understood that at least aspects of the described embodiments may be combined or modified.

Although selected embodiments of the present invention have been shown and described, it is to be understood that the present invention is not limited to the described embodiments. Instead, it is to be appreciated that changes may be made to these embodiments without departing from the principles and spirit of the invention, the scope of which is defined by the claims and the equivalents thereof.

1. A formulation for reducing bacteria to reduce malodor, the formulation comprising:
between about 10% and 50% by weight of silica or sodium metaphosphate;
between about 10% and 30% by weight of glycerol;
between about 3% and 40% by weight of soap;
between about 0.1% and 20% by weight of chlorhexidine digluconate;
between about 0.5% and 3% by weight of sodium carboxymethylcellulose (“SCMC”);
between about 0.05% and 0.5% by weight of sodium saccharine;
between about 1% and 3% by weight of menthol;
not more than about 1% by weight of titanium dioxide;
between about 0.1% and 0.5% by weight of propyl parabens; and
distilled water to total 100% by weight.

2. The formulation for reducing bacteria of claim 1, wherein the formulation comprises:
about 40% by weight of silica or sodium metaphosphate;
about 23% by weight of glycerol;
about 5% by weight of soap;
about 0.1% by weight of chlorhexidine digluconate;
about 1.5% by weight of sodium carboxymethyl cellulose (“SCMC”);
about 1% by weight of sodium saccharide;
about 1.5% by weight of menthol;
about 0.5% by weight of titanium dioxide;
about 0.1% by weight of propyl parabens; and
about 27.3% by weight of distilled water.

3. A method for reducing oral malodor of a user, the method comprising:
delivering the formulation for reducing bacteria of claim 1 in either solid or liquid form to the oral cavity of a user with a delivery device containing the formulation for reducing bacteria such that the formulation for reducing bacteria causes reduction of bacterial colonies within the oral cavity;
coating and adhering to constituent members of the oral cavity with the formulation for reducing bacteria by use of the delivery device containing the formulation for reducing bacteria; and
optionally removing the formulation for reducing bacterial from the oral cavity.

4. A method for reducing oral malodor of a user, the method comprising:
delivering the formulation for reducing bacteria of claim 2 in either solid or liquid form to the oral cavity of a user with a delivery device containing the formation for reducing bacteria such that the formulation for reducing bacteria causes reduction of bacterial colonies within the oral cavity;
coating and adhering to constituent members of the oral cavity with the formulation for reducing bacteria by use of the delivery device containing the formulation for reducing bacteria; and
optionally removing the formulation for reducing bacteria from the oral cavity.

5. The method for reducing oral malodor of claim 3, wherein the soap has a pH between about 6.5 and 8.5.

6. The method for reducing oral malodor of claim 5, wherein the soap has a pH between about 7 and 8.

7. The method for reducing oral malodor of claim 6, wherein the soap has a pH of about 7.

8. The method for reducing oral malodor of claim 4, wherein the soap has a pH between about 6.5 and 8.5.

9. The method for reducing oral malodor of claim 8, wherein the soap has a pH between about 7 and 8.

10. The method for reducing oral malodor of claim 9, wherein the soap has a pH of about 7.

11. The method for reducing oral malodor of claim 3, wherein the delivery device is selected from the group consisting of an aerosol can, bottle, and toothbrush.

12. The method for reducing oral malodor of claim 4, wherein the delivery device is selected from the group consisting of an aerosol can, bottle, and toothbrush.

13. The method for reducing oral malodor of claim 3, wherein the form of the formulation for reducing bacteria is selected from the group consisting of a paste, pellet, powder or granule, bar, aerosol, liquid, and stick.

14. The method for reducing oral malodor of claim 4, wherein the form of the formulation for reducing bacteria is selected from the group consisting of a paste, pellet, powder or granule, bar, aerosol, liquid, and stick.

15. The method for reducing oral malodor of claim 3, wherein the formulation for reducing bacteria is applied after the cleaning of the oral cavity using a toothbrush and toothpaste, or using mouthwash.

16. The method for reducing oral malodor of claim 4, wherein the formulation for reducing bacteria is applied after the cleaning of the oral cavity using a toothbrush and toothpaste, or using mouthwash.

17. An external formulation for reducing bacteria to reduce epidermal malodor of a human or animal, the external formulation for reducing bacteria comprising: soap, water, not more than about 0.2% chlorhexidine digluconate, and not more than about 2% zinc pyrithione.

18. (canceled)

19. A method for reducing epidermal malodor of a user or an animal, the method comprising:
delivering the external formulation for reducing bacteria of claim 17 in either solid or liquid form to the epidermis of a user or an animal with a delivery device containing the external formulation for reducing bacteria such that the external formulation for reducing bacteria reduces colonies on the epidermis of the user or animal; and
coating and adhering to the epidermis of the user or the animal with the external formulation for reducing bacteria by use of the delivery device containing the external formulation for reducing bacterial.

20. (canceled)

21. The method for reducing epidermal malodor of claim 19, wherein the soap has a pH between about 6.5 and 8.5.

22. The method for reducing epidermal malodor of claim 21, wherein the soap has a pH between about 7 and 8.

23. The method for reducing epidermal malodor of claim 22, wherein the soap has a pH of about 7.

24. (canceled)

25. (canceled)

26. (canceled)

27. The method for reducing epidermal malodor of claim 23, wherein the delivery device is selected from the group consisting of a roll-on bottle, bar, or aerosol can.

28. (canceled)

29. An internal formulation for reducing bacteria in a nasal cavity of a user, the internal formulation for reducing bacteria comprising: soap, water, and not more than about 0.2% chlorhexidine digluconate.

30. A method for reducing microbial growth in a nasal cavity of a user, the method comprising:
delivering the internal formulation for reducing bacteria of claim 29 in liquid form to the nasal cavity of a user with a delivery device containing the internal formulation for reducing bacteria, such that the internal formulation for reducing bacteria reduces microbial growth and/or bacterial colonies within the nasal cavity of the user;
coating and adhering to the inner lining of the nasal and sinus cavity of the user with the delivery device containing the internal formulation for reducing bacteria; and
rinsing the nasal cavity with water such that the internal formulation for reducing bacteria is removed from the nasal cavity of the user.

31. The method for reducing microbial growth of claim 30, wherein the soap has a pH of about 7.
